(19) United States
(12) Patent Application Publication
(10) Pub. No.: US 2009/0110680 A1
(43) Pub. Date: Apr. 30, 2009

(54) Combination of an anti ED-B fibronectin domain antibody and gemcitabine

(76) Inventors: STEPHANIE SIEGER, BERLIN (DE); DIETMAR BERNDORFF, BERLIN (DE); LUDGER DINKELBORG, BERLIN (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/783,285

(22) Filed: Apr. 6, 2007

Related U.S. Application Data
(60) Provisional application No. 60/796,865, filed on May 3, 2006.

(30) Foreign Application Priority Data
Apr. 7, 2006 (EP) 06090051.1

(51) Int. Cl. A61K 39/395

(52) U.S. Cl. 424/134.1

(57) ABSTRACT

Disclosed is a combination of an anti edb fibronectin domain antibody and gemcitabine.

This application claims the benefit of U.S. Ser. No. 60/796,865, filed May 3, 2006, which is incorporated by reference herein.

The invention relates to a combination (i) of a fusion protein, comprising an εₛ₂-CH4 domain part and an antibody part, specifically recognising the extra domain B of fibronectin (ED-B-fibronectin), and (ii) and Gemcitabine and its use for treatment of cancer, in particular pancreatic cancer.

### STATE OF THE ART

Adenocarcinoma of the pancreas represents the fifth leading cause of cancer related death in industrialized Western Countries. (Parker S L, Tong T, Bolden S, Wingo P A. Cancer statistics, 1997. CA Cancer J Clin 1997; 47:5-27). The prognosis of patients diagnosed with pancreatic cancer is extremely poor with an estimated overall 5-year survival rate of only 1-4%. Surgical resection provides the only potentially curative treatment, but locally extended or metastasized disease precludes surgical treatment in most cases. (Rosewicz S, Wiedenmann B. Pancreatic carcinoma. Lancet 1997; 349:485-489; Cohen S J, Pinover W H, Watson J C, Meropol N J. Pancreatic cancer. Curr Treat Options Oncol 2000; 1:375-386). Moreover currently available palliative strategies have little impact on the aggressive course of this neoplasm, achieving objective response rates of less than 20% with a dismal median survival of 4-6 months. (Burris H A, III, Moore M J, Andersen J, Green M R, Rothenberg M L, Modiano M R, Cripps M C, Portenoy R K, Storniolo A M, Tarassoff P, Nelson R, Dorr F A, Stephens C D, Von Hoff D D. Improvements in survival and clinical benefit with gemcitabine as first-line therapy for patients with advanced pancreas cancer: a randomized trial. J Clin Oncol 1997; 15:2403-2413.). Current chemotherapeutic agents and radiation treatments rely on the rapidly dividing nature of tumor cells, thereby suffering from poor selectivity. Unacceptable toxicities towards proliferating nonmalignant cells limit dose escalation and prevent the administration of a curative dose. Moreover most chemotherapeutic agents do not preferentially accumulate at the tumor site (Bosslet K, Straub R, Blumrich M, Czech J, Gerken M, Sperker B, Kroemer H K, Gesson J P, Koch M, Monneret C. Elucidation of the mechanism enabling tumor selective prodrug monotherapy. Cancer Res 1998; 58:1195-1201.).

One of the most selective oncofetal markers associated with neo-angiogenesis and tissue remodeling known so far represents the extra domain B (ED-B) of Fibronectin (FN) (Castellani P, Viale G, Dorcaratto A, Nicolo G, Kaczmarek J, Querze G, Zardi L. The fibronectin isoform containing the ED-B oncofetal domain: a marker of angiogenesis. Int J. Cancer. 1994 Dec. 1; 59(5):612-8. Erratum in: Int J Cancer 1995 Jul. 4; 62(1):118. FNs are high molecular-weight extracellular matrix (ECM) components abundantly expressed in a range of healthy tissues and body fluids. Various different FN isoforms can be generated due to alternative splicing at the level of the primary transcript. The ED-B, a small domain of 91 amino acids, which is identical in sequence in mouse and man, is usually absent in both plasma and tissue-fibronectin, except for some blood vessels of the regenerating endometrium and the ovaries. (Alessi P, Ebbinghaus C, Neri D. Molecular targeting of angiogenesis. Biochim Biophys Acta 2004; 1654:39-49; Viti F, Tarli L, Giovannoni L, Zardi L, Neri D. Increased binding affinity and valence of recombinant antibody fragments lead to improved targeting of tumoral angiogenesis. Cancer Res 1999; 59:347-352). However, it may become inserted in the fibronectin molecule during active tissue remodeling associated with neo-angiogenesis, thereby accumulating around the neo-vasculature and in the stroma of malignant tumors and in other tissues undergoing remodeling and angiogenesis. Recently, a number of good quality antibodies specific for the ED-B domain of fibronectin have been generated. In particular, the human single chain Fv antibody fragment scFv(L19), which displays a picomolar binding affinity for ED-B, has been verified to selectively target tumor neovasculature, both in experimental tumor models (Viti F, Tarli L, Giovannoni L, Zardi L, Neri D. Increased binding affinity and valence of recombinant antibody fragments lead to improved targeting of tumoral angiogenesis. Cancer Res 1999; 59:347-352.) and in patients with cancer (Santimaria M, Moscatelli G, Viale G L, Giovannoni L, Neri G, Viti F, Leprini A, Borsi L, Castellani P, Zardi L, Neri D, Riva P. Immunoscintigraphic detection of the ED-B domain of fibronectin, a marker of angiogenesis, in patients with cancer. Clin Cancer Res 2003; 9:571-579.).

The ED-B domain of fibronectin, a sequence of 91 amino acids identical in mice, rats and humans, which is inserted by alternative splicing into the fibronectin molecule, specifically accumulates around neovascular structures and represents a target for molecular intervention (Zardi et al. (1987) EMBO J. Vol.: 6, pages 2337-2342; Carnemolla et al. (1989) J. Cell Biol. Vol: 108, pages 1139-1148, further Castellani et al, (1994) Int. J. Cancer, Vol. 59, pages 612-618). Using the human recombinant antibody L19 directed to the ED-B domain, the possibility of in vivo neovasculature targeting has been demonstrated in different tumour models (Tarli et al. (1999) Blood, Vol.: 94, pages 192-198; Viti et al. (1999) Cancer Res. Vol.: 347). Monoclonal antibodies specifically recognising the ED-B-fibronectin domain are described in WO 97/45544.

Monoclonal antibody L19 is described in WO 99/58570.

WO 01/62298 on page 8, line 12 refers to the L19 VH and L19 VL domain sequences described in Pini et al. (1998) J. Biol. Chem. 273: 21769-21776. Pini et al. describes parts of the sequence of L19 in Table II on page 21772. L19 has the EMBL accession Number AJ 006113.

Tumours cannot grow beyond a certain mass without the formation of new blood vessels (angiogenesis), and a correlation between micro vessel density and tumour invasiveness has been reported for a number of tumours (Folkmann (1995) Nat. Med., Vol. 1, page 27). Molecules capable of selectively targeting markers of angiogenesis create clinical opportunities for the diagnosis and therapy of tumours and other diseases characterised by vascular proliferation, such as rheumatoid arthritis, diabetic retinopathy and age-related macular degeneration (O'Reilly et al. (1996) Nat. Med. Vol.: 2 page 689, further O'Reilly et al. (1997) Cell, Vol.: 88, page 277, further Friedlander et al. (1995) Science Vol.: 270, page 1500, further Pasqualini et al. (1997) Nat. Biotechnol. Vol.: 15 page 542, further Huang et al. (1997) Science, Vol.: 275, page 547, further Kim et al. (1993) Nature, Vol.: 362, page 841, further Schmidt-Erfurth et al. (1997) Br. J. Cancer, Vol.: 75, page 54).

The principles of radiotargeting for cancer therapy are described by Kassis A I (2005) in Expert Opin. Drug Delivery Vol.: 2(6), 981-991 and by Press O W et al (2000) in Seminars in Oncology Vol.:27, No. 6 (Suppl. 12), 62-73.

L19-SIP and its use for radioimmunotherapy are described in WO 03/076469. Berndorff et al (2005) Clin. Cancer Res. Vol.: 11 (19 Suppl.), 7053S-7063S described the use of ¹³¹I-L19-SIP as preferred antibody format for radioimmunotherapy of solid tumours by targeting the extra domain B fibronectin. “SIP” stands for small immunoprotein. L19-SIP is an antibody format wherein the L19-scFv is linked to an εs2-CH4 domain, and wherein two monomeric chains form a homodimer covalently linked by an S—S bridge (see e.g. WO03/076469; Borsi et al., 2002, Int. J. Cancer, 102:28:534-539). CH4 is the domain that allows dimerization in the IgE molecule and the εs2-isoform contains a cysteine at the carboxyterminal end, which stabilizes the IgE-dimer through an inter-chain disulphide bond. In the final SIP molecule of L19-SIP the ScFv(L19) is connected to the εs2-CH4 domain by a GGSG linker. The disclosure of WO03/076469 is included by reference).

The radiotherapy of pancreatic cancer using ¹³¹I-L19 SIP in combination with Gemcitabine was disclosed on the Symposium: Medicine in psychiatric diseases, Oct. 16, 2005.

Pancreatic cancer is a chemoresistant cancer, and stand alone radiation therapy of pancreatic cancer does not result in a survival benefit for the patient. Several Clinical trials describe the combination of radiation therapy plus gemcitabine (Blackstock A. W. et al. J. Clin. Oncol. 17:2208-2212, 1999; Mose S. et al. Strahlenther Onkol. Vol.: 178, pages 59-70, 2002). The use of gemcitabine as radiosensitizer in combination with radioimmunotherapy with antibodies for the treatment of pancreatic cancer was investigated in several animal models. E.g. Gold D V et al. (Clin. Can. Res., Vol.: 9, 3929s-3937s, 2003) describe the combination of ⁹⁰Y and ¹³¹I labelled monoclonal antibodies against MUC1 mucin in combination with Gemcitabine, where the combined treatment resulted in significantly improved treatment efficacies.

There is therefore a strong medical need for a medicament to effectively treat pancreatic cancer. The present invention makes available novel and effective medicaments, which are suitable for the treatment of pancreatic cancers.

The present invention relates to a combination comprising at least a fusion protein and gemcitabine,

wherein the fusion protein in its monomeric form comprises an εₛ₂-CH4 domain part and

an antibody-part specifically recognising the ED-B-fibronectin domain.

The term “Gemcitabine” is to be understood as 2′-deoxy-2′,2′-difluorocytidine as well as physiologically acceptable salts thereof, in particular the hydrochloride salt thereof. The hydrochloride salt of 2′-deoxy-2′,2′-difluorocytidine is commercially available under the trade name Gemzar.

Gemcitabine in its base form has the formula

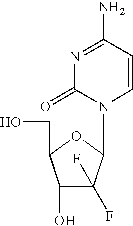

EP 0 122 707 discloses nucleosides containing difluor-carbohydrate groups for treatment of viruses. EP 0 184 365 describes the use of gemcitabine (1-(4-Amino-2-oxo-1H-pyrimidin-1-yl)-2-desoxy-2,2-difluororibose or 2′-deoxy-2′,2′-difluorocytidine) as a medicament for the treatment of susceptible neoplasms. The synthesis of gemcitabine is described e.g. in EP 0 184 365 and EP 727 433. The term “Gemcitabine” is to be understood as 2′-deoxy-2′,2′-difluorocytidine as well as physiologically acceptable salts thereof, in particular the hydrochloride salt thereof. The hydrochloride salt of 2′-deoxy-2′,2′-difluorocytidine is commercially available under the trade name Gemzar.

As described in the examples, it was found that the ED-B-fibronectin domain expression could be detected in pancreatic cancer tissue, but not in pancreatitis or normal pancreas. ED-B-fibronectin domain targeted radiotherapy experiments with 74 MBq ¹³¹I-L19-SIP resulted in tumour growth inhibition compared to untreated control tumours, demonstrated by a median survival of 44 days for mice that received a single dose of 74 MBq ¹³¹I-L19-SIP alone.

Moreover, it was surprisingly it was found that the combination comprising at least a fusion protein and gemcitabine, wherein the fusion protein in its monomeric form comprises an εₛ₂-CH4 domain part and an antibody-part specifically recognising the ED-B-fibronectin domain can be used much more successfully for treatment of cancer than either the fusion-protein alone or gemcitabine alone.

The fusion protein is present in dimeric form, wherein the monomers are linked by a S—S bridge, as described in Borsi et al., Int. J. Cancer, 2002, 102: 534-539.

In a preferred embodiment, the fusion protein in its monomeric form has a N-terminal antibody-part and C-terminal εₛ₂-CH4 domain part or wherein the fusion protein in its monomeric form has a N-terminal εₛ₂-CH4 domain part and a C-terminal antibody-part.

Especially preferred is the embodiment, wherein the fusion protein in its monomeric form has a N-terminal antibody-part and C-terminal εₛ₂-CH4 domain part.

In another preferred embodiment, the fusion protein in its monomeric form comprises a linker connecting the antibody-part and εₛ₂-CH4 domain part.

Preferably, the antibody-part is human.

In a preferred embodiment, the antibody-part specifically recognising the ED-B domain of fibronectin binds with sub-nanomolar affinity. For a review on the definitions and measurements of anti body-antigen affinity, see Ned et al. (1996). Trends in Biotechnol. 14, 465-470.

In a preferred embodiment, the antibody-part contains at least one of the CDRs of L19 antibody, preferably it contains all CDR sequences of the L19 antibody.

In a preferred embodiment, the antibody-part comprises at least one of the SEQ ID. No. 6 to 11. Preferably the antibody comprises the sequences according to SEQ ID no. 6 to 11.

In another preferred embodiment, the antibody-part comprises at least one V heavy chain according to Seq. Id. No. 01 or at least one V light chain according to Seq. Id. No. 02. In a more preferred embodiment, the antibody part comprises at least one least one V heavy chain according to Seq. Id. No. 01 and at least one V light chain according to Seq. Id. No. 02. In an even more preferred embodiment, the antibody-part comprises one V heavy chain according to Seq. Id. No. 01 and one V light chain according to Seq. Id. No. 02.

Preferred is an antibody part, wherein the heavy and the light chain are connected by an antibody linker.

In a preferred embodiment, the antibody linker comprises a sequence according to Seq. Id. No. 03, or a sequence having at least 90% identity to the sequence according to Seq. Id. No. 03. In a more preferred embodiment, the antibody linker has a sequence according to Seq. Id. No. 03.

In a more preferred embodiment, the εₛ₂-CH4 domain part comprises a sequence according to the Seq. Id No 04.

Preferred is the use of the fusion protein in its monomeric form according to the invention, wherein the fusion protein linker comprises a sequence according to the Seq. Id No 05, preferably the fusion protein linker has a sequence according to the Seq. Id No 05.

In a preferred embodiment the fusion protein linker has a length of 0 to 15 amino acids, more preferred 1 to 10, even more preferred 1 to 6 amino acids.

Preferred is a combination according to the invention, wherein the fusion protein is conjugated to a radioisotope. Methods for labelling fusion proteins of the invention are disclosed in Berndorff et al., Clin. Cancer Res., 2005; 11 (Suppl.), p. 7053s-7063s.

In a preferred embodiment, the radioisotope is a radioisotope selected from I; Tc, Re, In, Y, Lu, or I or a mixture thereof.

In an even more preferred embodiment, the radioisotope is selected from ¹²³I, ¹²⁴I, ¹²⁵I, ¹³¹I, ^{99m}Tc, ¹⁸⁸Re, ¹⁸⁸Re ²⁰³Pb, ⁶⁷Ga, ⁶⁸Ga, ⁴³Sc, ⁴⁷Sc, ^{110m}In, ¹¹¹In, ⁹⁷Ru, ⁶²Cu, ⁶⁴Cu, ⁶⁷Cu, ⁶⁸Cu, ⁸⁶Y, ⁹⁰Y, ⁸⁸Y, ¹²¹Sn, ¹⁵³Sm, ¹⁶⁶Ho, ¹⁰⁵Rh, ¹⁷⁷Lu, ¹⁷²Lu and/or ¹⁸F or a mixture thereof.

Most preferred is the use of ¹³¹I.

In an especially preferred embodiment of the present invention, an ¹³¹I-labeled fusion protein is used, wherein the fusion protein in its monomeric form contains a Vl domain of L19, a Vh domain of L19 and an εₛ₂-CH4 domain.

Most preferred is the use of ¹³¹I labelled L19-SIP.

In an especially preferred embodiment, the combination comprises ¹³¹I labelled L19-SIP and Gemcitabine, in particular Gemcitabine hydrochloride.

Preferred is a combination according to the invention for use as medicament.

More preferred is a combination according to the invention for use as a medicament for treatment of cancer.

In another embodiment, the invention relates to a combination according to the invention, wherein the cancer is selected from non-small cell lung cancer, head and neck cancer, and breast cancer, preferably pancreatic cancer.

“Specifically recognising” according to the present invention refers to antibody binding to a predetermined antigen. Typically, the antibody binds with an affinity of at least about 1×10⁷ M⁻¹, and binds to the predetermined antigen with an affinity that is at least two-fold greater than its affinity for binding to a non-specific antigen (e.g., BSA, casein) other than the predetermined antigen or a closely-related antigen. The antigen is the ED-B domain of fibronectin.

Antibody linker is any linker, preferably a peptide linker, which is suitable for linking Vh and Vl domains. Suitable linkers are for example described in Bird et al, Science, 242, 423-426, 1988; Huston et al, PNAS USA, 85, 5879-5883, 1988, EP 0 573 551; EP 0 623679 and EP 0 318554, which documents are introduced by reference.

Fusion protein linkers are linkers suitable for linking an antibody part according to the invention to the εₛ₂-CH4 domain. A suitable linker is shown in SEQ ID No. 5.

ε_{S2}-CH4 part is defined in Erqiu Li et al. “Mammalian cell expression of dimeric small immune proteins (SIP)” (1997) Protein Engineering Vol.: 10, no. 6 pages 731-736. The sequence of is shown in SEQ ID No. 4.

“Antibody-dependent cell-mediated cytotoxicity” and “ADCC” refers to a cell-mediated reaction in which non-specific cytotoxic cells that express Fc receptors (FcRs) (e.g. Natural Killer (NK) cells, neutrophils, and macrophages) recognise bound antibody on a target cell and subsequently cause lysis of the target cell. The primary cells for mediating ADCC, NK cells, express FcyRIII only, whereas monocytes express FcγRI, FcγRII and FcγRIII. FcR expression on haematopoietic cells in summarized is Table 3 on Page 464 of Ravetch and Kinet, Annu. Rev. Immunol 9.457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95.652-656 (1998).

“Human effector cells” are leukocytes which express one or more FcRs and perform effector functions. Preferably, the cells express at least FcγRIII and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See Daeron, Annu. Rev. Immunol. 15:203-234 (1997)). FcRs are reviewed in Ravetch and Kinet, Annu. Rev. Immunol 9:457-92 (1991), Capel et al., Immunomethods 4:25-34 (1994), and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the foetus (Guyer et al., J. Immunol. 11 7587 (1976) and Kim et al., J. Immunol. 24.249 (1994)).

The Antibody-part according to the present invention is understood as single chain Fv antibody fragment (scFv) of an antibody. Preferably, the antibody part is human, chimeric or humanized, particularly preferred human.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂ and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain Portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (See Kabat et al., Sequences of Proteins Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a Single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′), antibody fragments originally were produced as pairs of Fab′₂-fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ respectively. The subunit structures and three-dimensional configurations of different classes of immunoglobulins are well known.

“Single-chainFv” or “scFv” antibody fragments comprise the V_{H} and V_{L} domains of antibody, wherein these domains are present in a Single Polypeptide chain. Preferably, the Fv Polypeptide further comprises a Polypeptide linker between the V_{H} and V_{L}, domains which enables the scFv to form the desired structure for antigen binding. For a review of scFv See Pluckthun in The Pharmacology of Monoclonal Antibodies, vol. 113, Rosenburg and Moore eds., Springer-Verlag, New York, pp. 269-315 (1994).

In a further embodiment, antibodies or antibody fragments can be isolated from anti-body phage libraries generated using the techniques described in McCafferty et al., Nature, 348552-554 (1990). Clackson et al., Nature, 352:624-628 (1991) and Marks et al., J. Mol. Biol., 222581-597 (1991) describe the isolation of murine and human antibodies, respectively, using phage libraries. Subsequent publications describe the production of high affinity (mM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies. The DNA also may be modified, for example, by substituting the coding sequence for human heavy- and light-chain constant domains in place of the homologous murine sequences (U.S. Pat. No. 4,816,567; Morrison, et al., Proc. Nat. Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 0 404 097; WO 93/11161; and Hollinger et al., Proc. Natl. Acad. Sci. USA, 905444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a Single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal anti-bodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as sequiring production of the antibody by any particular method. For example, the monoclonal antibodies to be used in accordance with the present invention may be made by the hybridoma method first described by Kohler et al., Nature, 256: 495 (1975), or may be made by recombinant DNA methods (See, e.g., U.S. Pat. No. 4,816,567).

The “monoclonal antibodies” may also be isolated from phage antibody libraries using the techniques described in Clackson et al., Nature, 352:624-628 (1991) and Marks et al., J. Mol. Biol., 222581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,516,567; Morrison et al., Proc. Natl. Acad. Sci. USA, 81:6851-6855 (1984)).

Chimeric antibodies of interest herein include “primatised” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

Humanised antibodies: Methods for humanising non-human antibodies have been described in the art. Preferably, a humanised antibody has one or more amino acid residues introduced into it from a source which is non-human. These non-human amino acid residues are often referred to as “import” residues, which are typicality taken from an “import” variable domain. Humanisation can be essentially performed following the method of Winter and Co-workers (Jones et al., Nature, 321522-525 (1986); Riechmann et al., Nature, 332:323-327 (1988); Verhoeyen et al., Science, 239: 1534-1536 (1988)), by substituting hypervariable region sequences for the corresponding sequences of a human antibody. Accordingly, such “humanised” antibodies are chimeric antibodies (U.S. Pat. No. 4,816,567) wherein substantially less than an intact human variable domain has been substituted by the corresponding sequence from a non-human species. In practice, humanised antibodies are typically human antibodies in which some hypervariable region residues and possibly some FR residues are substituted by residues from analogous sites in rodent antibodies. The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanised antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The Same framework may be used for several different humanised antibodies (Carter et al., Proc. Natl. Acad. Sci. USA, 89:4285 (1992); Presta et al., J. Immunol., 151:2623 (1993)). It is further important that antibodies be humanized with retention of high affinity for the antigen and other favourable biological properties. To achieve this goal, according to a preferred method, humanised antibodies are prepared by a process of analysis of the parental sequences and various conceptual humanised products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences.

Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human antibodies: As an alternative to humanisation, human antibodies can be generated. For example, it is now possible to produce transgenic animals (e.g., mice) that are capable, upon immunisation, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous anti-body production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review See, e.g., Johnson, Kevin S, and Chiswell, David J., Current Opinion in Structural Biology 3:564-571 (1993). Several sources of V-gene Segments can be used for phage display. Clackson et al., Nature, 352:624-628 (1991) isolated a diverse array of anti-oxazolone antibodies from a small random combinatorial library of V genes derived from the Spleens of immunized mice. A repertoire of V genes from unimmunized human donors can be constructed and antibodies to a diverse array of antigens (including self-antigens) can be isolated essentially following the techniques described by Marks et al., J. Mol. Biol. 222581-597 (1991), or Griffith et al., EMBO J. 12:725-734 (1993). See, also, U.S. Pat. Nos. 5,565,332 and 5,573,905. Human antibodies may also be generated by in vitro activated B cells (see U.S. Pat. Nos. 5,567,610 and 5,229,275).

The term “hypervariable region” when used herein refers to the amino acid residues of an antibody which are responsible for antigen-binding. The hypervariable region comprises amino acid residues from a “complementarily determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

Antibody fragments: Various techniques have been developed for the production of antibody fragments. Traditionally, these fragments were derived via proteolytic digestion of intact antibodies (See, e.g., Morimoto et al., Journal of Biochemical und Biophysical Methods 24: 107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Biotechnology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

The term “specifically recognising” refers to antibody binding to a predetermined antigen. Typically, the antibody binds with an affinity of at least 1×10⁷ M, and binds to the ED-B domain of fibronectin that is at least two-fold greater than the affinity for binding to a non-specific antigen (e.g. BSA, casein) other than ED-B domain of fibronectin.

Amino acid sequence modification(s) of protein or Peptide antagonists or antibody-part described herein are contemplated. For example, it may be desirable to improve the binding affinity and/or other biological properties of the antagonist. Amino acid sequence variants of the antagonist are prepared by introducing appropriate nucleotide changes into the antagonist nucleic acid, or by peptide synthesis. Such modifications include, for example, deletions from, and/or insertions into and or Substitutions of, residues within the amino acid sequences of the antagonist. Any combination of deletion, insertion, and substitution is made to arrive at the final construct, provided that the final construct possesses the desired characteristics. Therefore, in the case of the heavy of light chain a variation of 1; 2; 3; 4; 5; 6; 7; 8; 9; 10; 11; 12; 13; 14; 15; 16; 17; 18; 19: or 20 amino acids can be executed. In the case of ε_{S2}-CH4 a variation of 1; 2; 3; 4; 5; 6; 7; 8; 9; 10; 11; 12; 13; 14; 15; 16; 17; 20; 21; 22; 23; 24; 25; 26; 27; 28; 29; 30; 31; 32; or 33 amino acids can be executed. In the case of a linker a variation of 1; 2; 3; 4; 5; 6; or 7 amino acids can be executed. In case of the linker the variations are much more flexible, because function is simple to create a sufficient space between the functional amino acid sequences. A variation is defined as a deletion, insertion and/or substitution.

The amino acid changes also may alter post-translational processes of the antagonist, such as changing the number or position of glycosylation sites. A useful method for identification of certain residues or regions of the antagonist that are preferred locations for mutagenesis is called “alanine scanning mutagenesis” as described by Cunningham and Welk Science, 244: 1081-1085 (1989). Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed antagonist variants are screened for the desired activity. Amino acid sequence insertions include amino- and/or carboxyl-terminal fusions ranging in length from one residue to Polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide which increases the serum half-life of the antagonist. Another type of variant is an amino acid substitution variant. These variants have at least one amino acid residue in the antagonist molecule replaced by a different residue. The sites of greatest interest for substitutional mutagenesis of antibody antagonists include the hypervariable regions, but FR alterations are also contemplated. Conservative substitutions are shown in Table 1 under the heading of “preferred substitutions”.

TABLE 1

| Original | | Preferred |
|---|---|---|
| Residue | Exemplary substitution | Substitution |
| Ala | Val; Leu; Ile | Val |
| Arg | Lys, Gln, Asn | Lys |
| Asn | Gln, His, Asp, Lys, Arg | Gln |
| Asp | Glu, Asn | Glu |
| Cys | Ser, Ala | Ser |
| Gln | Asn, Glu | Asn |
| Glu | Asp, Gln, | Asp |
| Gly | Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile, | Leu |
| Phe | Leu, Val, Ile, Ala, Tyr | Tyr |
| Pro | Ala | Ala |
| Ser | Thr | Thr |
| Thr | Ser | Ser |
| Trp | Tyr, Phe | Tyr |
| Tyr | Trp, Phe, Thr, Ser | Phe |
| Val | Ile, Leu, Met, Phe, Ala, Norleucine | Leu |

If such substitutions result in a change in biological activity, then more substantial changes, denominated “exemplary substitutions” in Table 1, or as further described below in reference to amino acid classes, may be introduced and the products screened. Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Norleucine, Met, Ala, Val, Leu, Ile;

(2) neutral hydrophilic: Cys, Ser, Thr;

(3) acidic: Asp, Glu;

(4) basic: Asn, Gln, His, Lys, Arg;

(5) residues that influence chain orientation: Gly, Pro; and

(6) aromatic: Trp, Tyr, Phe.

Non-conservative substitutions will entail exchanging a member of one of these classes for another class. Any cysteine residue not involved in maintaining the proper conformation of the antagonist also may be substituted, generally with serine, to improve the oxidative stability of the molecule and prevent aberrant cross linking. Conversely, cysteine bond(s) may be added to the antagonist to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyse a crystal structure of the antigen-antibody complex to identify contact points between the antibody and antigen. Such contact residues and neighbouring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. By altering is meant deleting one or more carbohydrate moieties found in the antagonist, and/or adding one or more glycosylation sites that are not pre-sent in the antagonist.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist is conveniently accomplished by altering the amino acid sequence such that it contains one or more of the above-described tri-peptide sequences (for N-linked glycosylation sites). The alteration may also be made by the addition of, or substitution by, one or more serine or threonine residues to the sequence of the original antagonist (for O-linked glycosylation sites).

Nucleic acid molecules encoding amino acid sequence variants of the antagonist are pre-pared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

It may be desirable to modify the antagonist of the invention with respect to effector function, e.g. so as to enhance antigen-dependent cell-mediated cyotoxicity (ADCC) and/or complement dependent cytotoxicity (CDC) of the antagonist. This may be achieved by introducing one or more amino acid substitutions in an Fc region of an anti-body antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumour activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

Variations of the fusion protein L19-SIP are defined by variations as mentioned before. That means the variations of special sequences, mentioned in the following list of Table 2, which can be modified be deletion, insertion and/or substitution by the following numbers of amino acids:

TABLE 2

| | | Numbers of amino acids, which may be |
|---|---|---|
| | | deleted, inserted and/or substituted |
| Seq. Id No | Type | compared to SEQ ID. No. 1, 2, 3, 4, and 5. |
| Seq. Id No 1 | VH | 1, 2, 3, 4, 5, 6, 7, 8, 9, 10, 11, 12, |
| | | 13, 14, 15, 16, 17, 18 |
| Seq. Id No 2 | VL | 1, 2, 3, 4, 5, 6, 7, 8, 9, 10, 11, 12, |
| | | 13, 14, 15, 16, 17, 18 |
| Seq. Id No 3 | mAb linker | 1, 2, 3; 4, 5, 6, 7 |
| Seq. Id No 4 | ε_{S2}-CH4 | 1, 2 ,3, 4, 5, 6, 7, 8, 9, 10, 11, 12, |
| | | 13, 14, 15, 16 17, 18, 19, 20, 21, 22, |
| | | 23, 24, 25, 26, 27, 28, 29, 30 |
| Seq. Id No 5 | Fusion pro- | 1, 2, 3 |
| | tein linker | |

Further modifications are mentioned in Pini et al. (1998) J. Biol. Chem., Vol.: 273, pages 21769-21776, WO 02/20563, WO 2005/37312, and WO 99/58570

Many appropriate imaging and radiotherapeutic agents are known in the art, as are methods for their attachment to antibody parts, fusion proteins, antibodies and binding ligand (see e.g., U.S. Pat. No. 5,021,236 and U.S. Pat. No. 4,472,509). Certain attachment methods involve the use of a metal chelate complex employing, for example, an organic chelating agent such a DTPA attached to the antibody (U.S. Pat. No. 4,472,509). Radioactively labelled fusion proteins and antibody parts can also be iodinated by contact with sodium or potassium iodide and a chemical oxidising agent such as sodium hypochlorite [Redshaw M. R., Lynch S. S., J. Endochrinol., 60, 527 (1974)], or an enzymatic oxidizing agent, such as lactoperoxidase [Marchalonis J. J., Biochem. J., 113, 299 (1969)]. The proteins of the inventions may be labelled with technetium-99 by a ligand exchange process, for example, by reducing pertechnetate with stannous solution, chelating the reduced technetium onto a sephadex column and applying the antibody to this column. Direct labelling techniques are also suitable, e.g., by incubation pertechnetate, a reducing agent such as SnCl₂, a buffer solution such as sodium-potassium phthalate solution, and the protein.

### Use as a Medicament for Radiotherapy/Chemotherapy

The combination of the invention exhibits pharmacological activity and is, therefore, useful as pharmaceuticals. In particular, the combination shows pharmacological activity in a number of pathological or disease states in connection with a cancer, especially pancreatic cancer.

The combination of the invention is therefore indicated for use in treatment of non-small cell lung cancer, head and neck cancer, colon and breast cancer, especially pancreatic cancer. The fusion protein and gemcitabine may be administered simultaneously or separately, at different times.

For the treatment of such conditions, the appropriated dosage will, of course, vary depending upon, for example, the host, the mode of administration and the nature and severity of the condition being treated.

The dosage of gemcitabine administered will vary according to the mode of use and route of use, as well as to the requirements of the patient. In general, a daily dose for a systemic therapy for an adult average patient will be about 10 to 150 mg/kg body weight/day, preferably 10 to 40 mg/kg/day. For once-weekly application the standard dose for gemcitabine is 1000-1250 mg/m² as an intravenous infusion over 30 minutes.

The dosage of the fusion protein, in particular radiolabelled L19-SIP, administered will vary according to the mode of use and route of use, as well as to the requirements of the patient. In general, a single dosage in man is in the range of about 2-200 MBq/kg body weight (10-1,000 μg/kg body weight). The radio-labelled fusion protein must be given in a dose that does not cause toxicity of the most radiosensitive organ in the body (dose limiting organ). In animal experiments the red bone marrow was identified as dose limiting organ for ¹³¹I labelled antibodies. For proteins labelled with a radio-metal the kidney can be the dose limiting organ.

The preferred method of administration is parenteral, in particular intravenous infusion.

In another embodiment, the invention relates to a method of treating cancer in a patient in need of such treatment which method includes the administration of a combination of the present invention.

In another embodiment, the invention relates to a method of treating cancer in a patient in need of such treatment which method includes the administration of at least one fusion protein of the invention and gemcitabine.

The fusion protein and gemcitabine may be administered together, or separately, at the same time points or at different time points, as described above. Also, a single administration of the combination is possible or administration of several doses.

SEQ ID No. 1 represents the Vl chain of the L19-SIP antibody part.

SEQ ID No. 2 represents the Vh chain of the L19-SIP antibody part.

SEQ ID No. 3 represents the antibody linker linking Vl and Vh the L19-SIP antibody part.

SEQ ID No. 4 represents the εₛ₂-CH4 domain part of L19-SIP

SEQ ID No. 5 represents the linker linking the antibody part of L19-SIP and the εₛ₂-CH4 domain.

SEQ ID No. 6 to 11 represent the CDR sequences of the L119.

SEQ ID No. 12 represents the sequence of L19-SIP in its monomeric form.

SEQ ID No. 13 represents the sequence of an alternative antibody or fusion protein linker.

### EXAMPLES

### Method of the Experiment

ED-B-fibronectin domain expression in mouse pancreatic tumour models as well in human pancreatic cancer tissue, pancreatitis and normal pancreas was investigated by immunohistochemical analysis. SPECT (=single photon emission computed tomography) imaging was performed in an orthotopic pancreas cancer mouse model (DAN-G human pancreas carcinoma, DMSZ No. ACC249) with the ^{99m}Tc labelled ED-B-fibronectin domain targeting scFv antibody fragment AP39, described in WO 03/055917. In addition MRI imaging was performed. In radiotherapy studies, mice bearing human pancreatic tumour xenografts (Capan-1, human pancreas adenocarcinoma, ATTC-No. HTB-79) were treated with a single dose of either 37 MBq or 74 MBq of ¹³¹I-labelled ED-B-fibronectin domain targeting L19-SIP (=fusion protein). For combination therapy Capan-1 tumour bearing mice received two cycles of gemcitabine (1 g/m²/week for three weeks with a one-week break), followed by a single dose of 37 MBq ¹³¹I-L19-SIP 24 h after the first injection of gemcitabine. In a first experiment Capan-1 tumour bearing mice received either a single dose of 37 MBq of ¹³¹I labelled ED-B-fibronectin domain targeting L19-SIP, or two cycles of gemcitabine (1 g/m²/week for three weeks with a one-week break) as stand alone therapies. For combination therapy mice received two cycles of gemcitabine together with a single dose of 37 MBq ¹³¹I-L19-SIP 24 h after the first injection of gemcitabine. In a second experiment Capan-1 tumour bearing mice were treated with a single dose of either 37 MBq or 74 MBq of ¹³¹I labelled ED-B-fibronectin domain targeting L19-SIP (=fusion protein). For combination therapy Capan-1 tumour bearing mice received two cycles of gemcitabine (1 g/m²/week for three weeks with a one-week break), together with a single dose of 37 MBq 1311-L19-SIP 24 h after the first injection of gemcitabine.

### Results of the Experiment

ED-B-fibronectin domain expression could be detected in pancreatic cancer tissue, but not in pancreatitis or normal pancreas. In contrast to MRI, SPECT-imaging using ^{99m}Tc-AP39 clearly visualized the orthotopic tumour. Fusion images of SPECT and MRI confirmed the tumour-restricted accumulation of ^{99m}Tc AP39 in the pancreas. ED-B fibronectin domain targeted radiotherapy experiments with 37 and 74 MBq ¹³¹I-L19-SIP did not result in tumour growth inhibition compared to untreated control tumours.

### The Synergistic Effect of the Combination of the Invention is Shown by the Following Test Results:

An improved median survival of 58 days was achieved in mice that received the combined treatment of a single dose of 37 MBq ¹³¹I-L19-SIP plus gemcitabine, or of 44 days for mice that received a single dose of 74 MBq ¹³¹I-L19-SIP alone, versus 32 days for untreated control animals.

Survival of mice either treated with 37 MBq ¹³¹I-L19-SIP alone or with gemcitabine alone, did not differ from untreated control animals (36 days).

ED-B-fibronectin domain targeted SPECT imaging significantly improved the detection of pancreatic cancer compared to MRI. Surprisingly, combined chemotherapy/EDB-fibronectin domain targeted radio immunotherapy regimen significantly increases anti-tumour efficacy compared to each treatment arm given alone. There results demonstrate the ED-B-fibronectin domain targeted imaging and combined chemotherapy/ED-B-fibronectin domain targeted radio immunotherapy are showing new approaches for the detection and treatment of pancreatic cancer, respectively.

1. A combination comprising at least (i) a fusion protein and (ii) gemcitabine, wherein the fusion protein in its monomeric form comprises
an antibody-part,
specifically recognising the ED-B-fibronectin domain and
an εₛ₂-CH4 domain part.

2. A combination according to claim 1,
wherein the fusion protein in its monomeric form has an N-terminal antibody-part and a C-terminal εₛ₂-CH4 part or
wherein the fusion protein in its monomeric form has an N-terminal εₛ₂-CH4 part and a C-terminal antibody-part.

3. A combination according to claim 1, wherein the fusion protein is present in dimeric form.

4. A combination according to claim 1, wherein the antibody-part binds specifically to the ED-B oncofetal fibronectin domain with sub-nanomolar affinity.

5. A combination according to claim 1, wherein the antibody-part contains at least one CDR sequence of the L19 antibody.

6. A combination according to claim 1, wherein the antibody-part comprises the sequences according to SEQ ID no. 6 to 11

7. A combination according to claim 1, wherein the antibody-part comprises at least one V heavy chain according to Seq. Id. No. 01 or at least one V light chain according to Seq. Id. No. 02.

8. A combination according to claim 1, wherein the antibody-part comprises one V heavy chain according to Seq. Id. No. 01 and one V light chain according to Seq. Id. No. 02.

9. A combination according to claim 1, wherein the heavy and the light chain are connected by an antibody linker.

10. A combination according to claim 1, wherein the antibody linker comprises a sequence according to Seq. Id. No. 03, or a sequence having at least 90% identity to the sequence according to SEQ. ID. No. 03.

11. A combination according to claim 1, wherein the εₛ₂-CH4 domain part is human.

12. A combination according to claim 1, wherein the εₛ₂-CH4 domain part comprises a sequence according to SEQ. ID. No. 04.

13. A combination according to claim 1, wherein a fusion protein linker is connecting the antibody-part and the εₛ₂-CH4 domain part.

14. A combination according to claim 1, wherein the fusion protein linker has a length of 1 to 6 amino acids.

15. A combination according to claim 1, wherein the fusion protein linker comprises a sequence according to SEQ ID No. 05.

16. A combination according to claim 1, wherein gemcitabine is gemcitabine hydrochloride.

17. A combination according to claim 1, wherein the anti ED-B fibronectin domain antibody is conjugated to a radioisotope.

18. A combination according to claim 17, wherein the radioisotope is selected from I; Tc, Re, In, Y, Lu, or I or a mixture thereof.

19. A combination according to claim 18, wherein the radioisotope is selected from ¹²³I, ¹²⁴I, ¹²⁵I, ¹³¹I, ^{99m}Tc, ¹⁸⁶Re ¹⁸⁸Re ²⁰³Pb ⁶⁷Ga, ⁶⁸Ga, ⁴³Sc, ⁴⁷Sc, ^{110m}In, ¹¹¹In, ⁹⁷Ru, ⁶²Cu, ⁶⁴Cu, ⁶⁷Cu, ⁶⁸Cu, ⁸⁶Y, ⁹⁰Y, ⁸⁸Y, ¹²¹Sn, ¹⁵³Sm, ¹⁶⁶Ho, ¹⁰⁵Rh, ¹⁷⁷Lu, ⁷²Lu and/or ¹⁸F or a mixture thereof.

20. A combination according to claim 19, wherein the radioisotope is selected from ¹³¹I.

21. A combination according to claim 20, wherein a ¹³¹I-labeled fusion protein is used, and wherein the fusion protein in its monomeric form contains a VI domain of L19, a Vh domain of L19 and an εₛ₂-CH4 domain.

22. A combination according to claim 21, wherein the fusion protein is ¹³¹I labelled L19-SIP

23. A combination according to claim 22, wherein the combination comprises ¹³¹I labelled L19-SIP and Gemcitabine.

24. A combination according to claim 1, for use as a medicament.

25. A combination according to claim 1, for use as a medicament for the treatment of cancer.

26. A combination according to claim 25, wherein the cancer is selected from non-small cell lung cancer, head and neck cancer, ovarian cancer and breast cancer.

27. A combination according to claim 26, wherein the cancer is pancreatic cancer.

28. A method of treating cancer in a patient in need of such treatment which method includes the administration of a combination according to claim 1.


SEQUENCE LISTING (13 sequences)

SEQ ID NO: 1 - protein, 116 aa, Artificial [sequence omitted]

SEQ ID NO: 2 - protein, 108 aa, Artificial [sequence omitted]

SEQ ID NO: 3 - protein, 12 aa, Artificial
  DOMAIN (1)..(12): Antibody linker region
GDGSSGGSGGAS

SEQ ID NO: 4 - protein, 116 aa, Artificial
  DOMAIN (1)..(116): etas2-CH4 domain
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 5 - protein, 5 aa, Artificial
  DOMAIN (1)..(5): fusion protein linker
SGGSG

SEQ ID NO: 6 - protein, 5 aa, Artificial
  DOMAIN (1)..(5): CDR1 of VH L19
SFSMS

SEQ ID NO: 7 - protein, 17 aa, Artificial
  DOMAIN (1)..(17): CDR2 of VH L19
SISGSSGTTYYADSVKG

SEQ ID NO: 8 - protein, 7 aa, Artificial
  DOMAIN (1)..(7): CDR3 of VH L19
PFPYFDY

SEQ ID NO: 9 - protein, 12 aa, Artificial
  DOMAIN (1)..(12): CDR1 of Vl L19
RASQSVSSSFLA

SEQ ID NO: 10 - protein, 7 aa, Artificial
  DOMAIN (1)..(7): CDR2 of Vl L19
YASSRAT

SEQ ID NO: 11 - protein, 9 aa, Artificial
  DOMAIN (1)..(9): CDR3 of Vl L19
QQTGRIPPT

SEQ ID NO: 12 - protein, 357 aa, Artificial
  DOMAIN (1)..(357): sequence of  L19-SIP in its monomeric form
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 13 - protein, 17 aa, Artificial
  DOMAIN (1)..(17): alternative antibody or fusion protein linker
EFSSSSGSSSSGSSSSG